(19) United States
(12) Patent Application Publication
(10) Pub. No.: US 2006/0009432 A1
(43) Pub. Date: Jan. 12, 2006

(54) Use of neurosteroids to treat neuropathic pain

(76) Inventors: Roger Whiting, Los Altos, CA (US); Lakshmi Sangameswaran, San Jose, CA (US)

Correspondence Address: FOLEY & LARDNER LLP, 1530 PAGE MILL ROAD, PALO ALTO, CA, 94304, US

(21) Appl. No.: 11/177,977

(22) Filed: Jul. 8, 2005

Related U.S. Application Data
(60) Provisional application No. 60/586,825, filed on Jul. 9, 2004.

(51) Int. Cl. A61K 31/57

(52) U.S. Cl. 514/177

(57) ABSTRACT

Ganaxolone (3α-hydroxy-3β-methyl-5β-pregnan-20-one) and 3α-hydroxy-3β-methyl-5β-pregnan-20-one and their physiologically cleavable esters are useful for treating neuropathic pain. Compositions comprising a pharmaceutically acceptable excipient and an appropriate compound, along with methods for preparing the compositions are disclosed. Also disclosed are articles of manufacture wherein the composition is combine with labeling instructions for treating neuropathic pain.

### CROSS-REFERENCE TO OTHER APPLICATIONS

This application claims priority to U.S. Provisional Application 60/586,825, filed on 9 Jul. 2004, which application is incorporated herein by reference.

### FIELD OF THE INVENTION

The present invention relates to methods, compositions, and articles of manufacture for treating neuropathic pain.

### BACKGROUND OF THE INVENTION

Neuropathic pain comprises spontaneous or stimulus-independent pain, which has been described as shooting, burning, lancinating, prickling and electrical, and evoked or stimulus-dependent neuropathic pain, which is principally characterized as allodynia and hyperalgesia. It is not a single disease entity, but rather includes a range of heterogeneous conditions that differ in etiology, location and initiating cause. A discussion of neuropathic pain can be found in “The Merck Manual”, Sixteenth Edition, 1992 (published by Merck Research Laboratories) at pages 1416-1417.

Neuropathic pain is often difficult to control. Mild pain may sometimes be alleviated by analgesics sold over the counter. Several classes of drugs have recently proved helpful to many patients suffering from more severe forms of chronic neuropathic pain. These include mexiletine, a drug developed to correct irregular heart rhythms (sometimes associated with severe side effects); several antiepileptic drugs, including gabapentin, phenyloin, and carbamazepine; and some classes of antidepressants, including tricyclics such as amitriptyline. Injections of local anesthetics such as lidocaine or topical patches containing lidocaine may relieve more intractable pain. In the most severe cases, doctors can surgically destroy nerves; however, the results are often temporary and the procedure can lead to complications.

Pain is not a symptom that exists alone. Other problems associated with pain include fatigue, sleeplessness, withdrawal from activity, increased need to rest, compromised immune system function, changes in mood including hopelessness, fear, depression, irritability, anxiety, and stress, and physical disability. There is no single drug that is reliably helpful in alleviating neuropathic pain. Thus, neuropathic pain represents a disorder with a high unmet medical need.

Ganaxolone is a neurosteroid that binds with a distinct site in the GABAA receptor protein potentiating its inhibitory action in neurotransmission. GABA_{A} receptors mediate a significant portion of the first inhibitory synaptic transmission in the central nervous system. In addition to neurosteroids such as ganaxolone, a number of compounds such as benzodiazepines, barbiturates and general anesthetics also bind with distinct sites in the GABAA receptor protein thereby acting as potent allosteric modulators of the receptor. Among the benzodiazepines (e.g., valium) and barbiturates (e.g., phenobarbital), there are well known antipileptics that have been used to treat a variety of seizures in the clinic. These compounds have demonstrated a significant efficacy in a variety of preclinical animal models of seizure activity. In addition, they are also known to be potent anxiolytics, muscle relaxants and sedatives. To this date, there is no documented evidence that these allosteric modulators of the GABA receptor protein have significant efficacy in pain models, both acute and neuropathic pain conditions.

Ganaxolone is a neurosteroid taught to be a possible anticonvulsant and antiepileptic with potential utility in the treatment of generalized absence seizures as well as sinple and complex partial seizures. See Carter, et al., G. Phar. And Exp. Ther., Vol. 280, #3, 1284-1295. Ganaxolone is also taught to be a positive allosteric modulator of GABA_{A}, but failed to show benefit on time to pain relief in a phase 2 clinical trial for migraine. See Current Medical Research and Opinion, Vol. 17, Suppl. 1, 2001, 574.

Alphadolone is another neurosteroid with GABA potentiating properties. It is an active component of Safin along with alphaxolone (also a neurosteroid and an anesthetic), prescribed in veterinary medicine for acute pain. Alphadolone, however, is inactive in the neuropathic pain model as disclosed in Example 2.

In light of these observations, it is surprising to find that ganaxolone has activity in treating neuropathic pain.

### SUMMARY OF THE INVENTION

One aspect of the invention provides a method of treating neuropathic pain comprising administering a therapeutically effective amount of a neurosteroid to a patient having neuropathic pain, wherein, the neurosteroid has the formula (I), as follows: 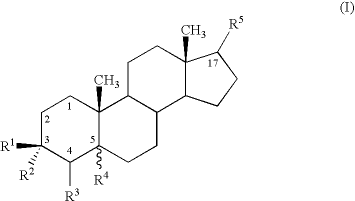
wherein R¹ is methyl, R² is hydroxyl or a physiologically cleavable ester thereof, R³ is hydrogen, R⁴ is alpha or beta hydrogen, and R⁵ is acetyl (CH₃C(O)—). R⁴ is preferably in the alpha-position to provide compounds having the formula (II):

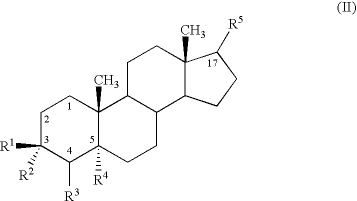

Another aspect of this invention provides a pharmaceutical composition suitable for treating neuropathic pain, which composition comprises a compound of formula (I) and a pharmaceutically acceptable excipient.

Another aspect of the invention provides an article of manufacture comprising (a) a composition comprising the above neurosteroid and a pharmaceutically acceptable excipient; and (b) a label with instructions for using the composition to treat neuropathic pain in a patient.

Yet another aspect of the invention provides a method for preparing a pharmaceutical composition useful for treating neuropathic pain, which method comprises (a) combining the above neurosteroid with a pharmaceutically acceptable excipient to form a formulation acceptable for administration to a human; and (b) packaging the formulation with written instructions for the treatment of neuropathic pain by administering the formulation to a patient in need of such treatment.

Another aspect of this invention is the use of the compound described above in the preparation of a composition useful for the treatment of neuropathic pain.

### DETAILED DESCRIPTION AND PRESENTLY PREFERRED EMBODIMENTS DEFINITIONS

In accordance with the present invention and as used herein, the following terms are defined with the following meaning, unless explicitly stated otherwise.

The term “physiologically cleavable ester” refers to a product of the hydroxyl of the neurosteroid of formula (I) and an acid or acid derivative, wherein the product is cleaved in the body to give the compound formula (I) or an active metabolite. Such a physiologically cleavable ester can be viewed as a “pro-drug.” Such a “pro-drug” is particularly valuable if it increases the bioavailability of the compound where R² is hydroxyl when such a pro-drug is administered to a subject. For example, a “pro-drug” administered orally may be more readily absorbed into the blood or may enhance delivery of the parent compound to a biological compartment of the subject such as the brain or lymphatic system. A general overview of pro-drugs is provided, l.a., in (1) “Pro-drugs As Novel Delivery Systems,” Vol. 14 of the ACS Symposium Series, by T. Higuchi and V. Stella or (2) “Bioreversible Carriers in Drug Design,” American Pharmaceutical Association, Porgamon Press, 1987, Edward B. Roche, Ed. Both references are incorporated herein by reference to aid one of skill in the art to prepare such physiologically cleavable esters or pro-drugs consistent with the teaching of this patent application. Such esters are well known in the art and include the esters of numerous physiological carboxylic acids. Such carboxylic acids include aliphatic, aromatic or hetero carboxylic acids, that may be unsubstituted or substituted.

Aliphatic carboxylic acids include both mono- and di-acids that are derived from lower alkyl, lower alkenyl and lower alkynyl entities and are characterized by the presence of one or two carboxyl groups, i.e., —C(O)OH.

The term “lower alkyl” carboxylic acid refers to a monovalent, saturated aliphatic hydrocarbon radical having from one to six of carbon atoms bonded to a carboxyl group. A “C₁₋₆ alkyl” or an “alkyl of 1-6 carbons” or “Alk 1-6” would refer to any alkyl group containing one to six carbons in the structure, respectively linked to a carboxyl group. Alkyl may be a straight chain (i.e. linear), a branched chain, or a cyclic structure. Representative examples of lower alkyl radicals include methyl, ethyl, n-propyl, n-butyl, n-pentyl, n-hexyl, isopropyl, isobutyl, isopentyl, amyl, sec-butyl, tert-butyl, tert-pentyl, cyclopropyl, cyclobutyl, and the like. The radical may be optionally substituted with substituents at positions that do not significantly interfere with the preparation of compounds falling within the scope of this invention and that do not significantly reduce the efficacy of the compounds. The alkyl may be optionally substituted with one to three substituents independently selected from the group consisting of halo, hydroxyl, cyano, nitro, or amino.

The term lower “alkenyl” carboxylic acid refers to an unsaturated aliphatic group that has 1-6 carbons and may be straight chain, branched chain, and cyclic groups, all of which may be optionally substituted similarly to the alkyl group. Representative examples of lower alkenyl radicals in carboxylic acids include vinyl (ethenyl), allyl (propen-3-yl), 1-buten-4-yl; 2-buten-4-yl, 1-penten-5-yl, and the like.

The term lower “alkynyl” carboxylic acid refers to unsaturated hydrocarbon groups which contain at least one carbon-carbon triple bond and includes straight chain and branched chain groups which may be optionally substituted. Suitable alkynyl groups include propyn-3-yl, pentyn-5-yl, and the like which may be optionally substituted similiarly to the alkyl group.

Aromatic carboxylic acids are those carboxylic acids characterized by the presence of at least one benzene ring or an entity that resembles benzene. Thus, such carboxylic acids include benzoic acid, which may be unsubstituted or substituted with a substituent that does not significantly reduce the efficacy, e.g., one to five lower alkyls, halo, hydroxyl, nitro, lower alkoxy, amino, cyano, and the like.

A hetero carboxylic acid is one that has a non-carbon atom such as N, S, or O linked to the carboxyl group. Thus, the hetero carboxylic acid is R⁶R⁷—N—C(O)OH, R⁸S—C(O)OH, or R⁹—O—C(O)OH and preferably is R⁶R⁷—N—C(O)OH. R⁶, R⁷, R¹ and R⁹ may each independently be lower alkyl, lower alkenyl, or lower alkynl each as defined herein) or R⁶ and R⁷ together with the N form a 5- or 6-member heterocyclic ring having zero one or two other non-carbon atoms in the ring such as N, S, or O. Examples of such heterocyclic rings include pyrrole, imidazole, pyrazole, 3-pyrroline, pyrolidine, pyridine pyrimidine, morpholine, and the like.

The term “neuroactive steroid” refers to an endogenous steroid (or its synthetic analog) that rapidly alters the excitability of neurons by direct actions on membrane ion channels, including GABA-A and NMDA receptors.

“Neuropathic pain” refers to pain initiated or caused by a primary lesion or dysfunction in the nervous system, which includes both situations in which there is actual injury, such as that due to nerve trauma or disease, and those in which there is pain in the absence of an actual physical lesion of the nerve. The latter includes complex regional pain syndrome Type I (formerly reflex sympathetic dystrophy) and some forms of trigeminal neuralgia.

The term “pharmaceutically-acceptable” means a moiety that is useful for forming pharmaceutical formulations and entities that are physiologically acceptable and generally non-toxic to a subject receiving the moiety.

Methods for Treating Neuropathic Pain

Compounds useful in this invention are those of formula (I), as defined herein. Ganaxolone (3α-hydroxy-3β-methyl-5α-prenan-20-one) is the preferred compound; while the corresponding 3α-hydroxy-3β-methyl-5β-pregnan-20-one is also useful. A published method of making ganaxalone is disclosed in Example 1. As mentioned hereinbefore, a physiologically cleavable ester of the 3-hydroxy group, especially of ganaxolone, is also useful. While the carboxylic acids from which such esters may be derived were generically mentioned previously, the following is a list of carboxylic acids useful to form the esters at the 3-position:
acetic acid,
n-propionic acid,
n-butyric acid,
t-butyl carboxylic acid,
n-pentanoic acid,
benzoic acid,
morpholinocarboxylic acid,
malonic acid,
succinic acid,
glutaric acid,
adipic acid,
pimelic acid,
suberic acid,
n-propenoic acid,
e-butenoic acid,
and the like.
   Other esters may be found by referring to U.S. Pat. No. 5,939,545, which is incorporated herein by reference.

In the treatment of neuropathic pain, the compositions of this invention may be administered by any suitable route which will introduce the intended compound into the bloodstream at a therapeutically effective amount. Thus, the mode of administration may be orally (including bucally), parenterally (e.g., intravenously (IV), intramuscularly (IM), subcutaneously (SC), transdermally, or any other acceptable route other than through the intestine), by suppository, and other routes that may be apparent to one of skill in the art.

Since prolonged use is anticipated, which may be for the entire remaining life of the patient, oral administration will be preferred for most patients. Preferred modes for oral ingestion include tablets, capsules, pills, powders, solutions, suspensions, emulsions, and the like that are swallowed, as well as transmembrane oral routes, such as lozenges, sublingual tablets or wafers, or chewing gum. Such preparations are all well known in the pharmaceutical arts, and generally include (in addition to the active agent) various types of carriers, diluents, and binders which can be molded or compressed into tablets, enclosed in gelatin capsules, mixed or suspended in a liquid syrup or emulsion, etc. Such compositions are preferably formulated or packaged in a unit dosage form, which refers to physically discrete units such as capsules or tablets, each unit containing a predetermined quantity of active material or materials calculated to produce the desired therapeutic effect, in association with a pharmaceutically acceptable carrier, such as a binding agent for compressed tablets or a liquid vehicle for syrups.

The preferred dosage of a chosen drug will depend upon both the potency of the drug and the status of the patient. The composition will need to be prescribed by a treating physician, who will take into account any relevant factors, such as the age and weight of the patient, the severity of the patient's symptoms, and the chosen route of administration.

Generally the preferred orally administered composition is a tablet, i.e. a pharmaceutical dosage form prepared by compressing or molding a composition containing the compound useful in this invention. A tablet may be coated or uncoated, preferably the former. If coated, it may be sugar-coated, film-coated, enteric-coated, and the like. The tablets may be round, oval, oblong, cylindrical, triangular, or other acceptable shapes. The ingredients of a tablet, along with the active compound, will include pharmaceutically acceptable excipients such as diluents, binders, lubricants, preservatives, disintegrants, coloring agents, flavors, sweetness, and the like. Examples of these excipients can be found in the standard publication Remington's Pharmaceutical Sciences, 19^{th} Edition, Mack Publishing Co., Easton, Pa.—1995 (“Remington's”). Techniques for preparing tablets will be found in detail in Remington's, which is incorporated herein by reference to aid one of skill in the art.

Another useful oral composition is a capsule, where the drug containing composition is enclosed in a hard or soft soluble container or shell of a suitable form of gelatin. Generally the capsules are of an oblong shape and maybe filled manually or automatically by machine. A detailed description of the contents and preparing such capsules can also be found in Remington's.

Depending on the dosage form and the administration route, the amount of the active compound in the composition to be administered will be sufficient to deliver the desired amount of active to the subject being treated to alleviate the neuropathic pain, i.e., a therapeutically effective amount. Thus another aspect of the invention is the use of a component of formula (I) to prepare a composition useful for the treatment of neuropathic pain. The compound is confined with an excipient to form an acceptable formulation then combined with a label providing instructions for administration.

Another aspect of this invention is a pharmaceutical composition suitable for treating neuropathic pain, which composition comprises a compound of formula (I) and a pharmaceutically-acceptable excipient. Generally the amount of the active compound will vary from about 1 milligram (mg) to about 500 mg per dosage unit, preferably about 2 mg-100 mg, and most preferably about 5 mg-50 mg. Depending on the size of the dosage form, the active may vary between about 1% to about 90% by weight, preferably less than 50% by weight.

Thus the percentage of the active may be, e.g., 1, 2, 3, 4, 5, 10, 20, 30, 40, 50 percent or any intermediate percentage or range as desired. By using a dosage form with the desired composition percentage, a doctor skilled in the art can administer enough to achieve about 0.1 mg/kilogram (kg) body weight in the subject to about 10 mg/kg, preferably about 0.1 mg/kg to about 5 mg/kg. The label that accompanies the dosage form will provide instructions for using the composition to treat neuropathic pain.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

The following examples are provided as a guide for a practitioner of ordinary skill in the art. The examples should not be construed as limiting the invention, as the examples merely provide specific methodology useful in understanding and practicing an embodiment of the invention.

### EXAMPLES

### Example 1

### Synthesis of Ganaxolone

Ganaxolone (3α-hydroxy-3β-methyl-5α-pregnan-20-one) is synthesized as described in U.S. Pat. No. 3,953,429, incorporated herein by reference, or in Hogenkamp, et. al. J. Med. Chem. 40:61-72, 1997 also incorporated herein by reference. In brief, a mixture of sodium hydride (17 mg.), trimethyl-sulphoxonium iodide (300 mg.) and dimethyl sulphoxide (2 ml.) is stirred under nitrogen at room temperature for 1 hr. 5α-pregnane-3,20-dione-20-ketal (3 g), is then added and the resulting mixture is stirred for a further 2 hr. and poured into water. The precipitated solid is collected by filtration, washed with water and dried over P₂O₅ in vacuo. Recrystallisation from acetone/petrol gives 3(R)-20,20-ethylenedioxy-5α-pregnane-3-spiro-2′-oxirane, as white needles.

A solution of the above 3(R)-20,20-ethylenedioxy-5α-pregnane-3-spiro-2′-oxirane in tetrahydrofuran (5 ml.) is added to a stirred suspension of lithium aluminium hydride (0.5 g.) in ether (15 ml.). The resulting mixture is refluxed for 2 hr. treated with saturated aqueous ammonium chloride and partitioned between water and ether. The organic layer is washed with water, dried (Na₂SO₄) and evaporated. Recrystallisation of the residue from acetone gives 20,20-ethylenedioxy-3β-methyl-5α-pregnan-3α-ol (m.p. 139°), as white needles.

A solution of 20,20-ethylenedioxy-3β-methyl-5α-pregnan-3α-ol in acetone (60 ml.) is treated with a solution of potassium dichromate (1.5 g.) in 2N-sulphuric acid (15 ml.) at room temperature for 2 hr. The mixture is then poured into water and the precipitated solid is collected by filtration, washed with water and dried over phosphorus pentoxide in vacuo. Recrystallisation from acetone-petroleum ether gives 3α-hydroxy-3β-methyl-5α-pregnan-20-one (m.p. 103°), obtained as white needles.

### Example 2

### Cold Allodynia in Chronic Constriction Injury Model

Unilateral sciatic nerve injury was produced under deep anesthesia with i.p injection of sodium pentobarbital (Mebumal, 60 mg/kg), as described by Bennett and Xie (Pain, 33, 87-107(1988)). The common sciatic nerve was exposed and freed for about 10 mm at mid-thigh level. Four ligatures (Ethicon, 4.0 plain gut) were tied loosely around the nerve with about 1-mm spacing creating a chronic constriction injury (CCl), and the incision was closed.

Brisk foot withdrawal in response to acetone application was measured based on the method described by Choi et al. The acetone bubble was gently brought in touch with the plantar surface around the center, and the acetone quickly spread over the central part of the plantar surface of the foot. Applications were made five times (once every 5 min) to each paw. The score for each application was recorded as follows: foot withdrawal was scored as positive (1) and lack of withdrawal as negative (O). The total score (0 to 5) was taken as index for cold sensitivity of the foot with a lower score. This approximates pain from a non-noxious stimulus, i.e. allodynia.

Cold allodynia response was measured 15, 30, 45, 60, 90 and 120 minutes after i.p. administration of ganaxolone (at 1 mg/kg, 3 mg/kg or 10 mg/kg) as well as prior to administration of the neurosteroid and prior to infliction of the neural injury. Table 1 provides the resulting measurement of cold allodynia response in groups of six rats per dosage level. The abbreviation “s.e.m.” means standard error mean.

TABLE 1

Average time to withdrawal (s.e.m.)

| | | |
|---|---|---|
| 1 mg/kg | 3 mg/kg | 10 mg/kg |

| Pre-CCI | | 20.00 | 20.00 | | 20.00 | |
|---|---|---|---|---|---|---|
| Pre-admin | | 5.69 (0.74) | 5.96 | (0.65) | 6.98 | (0.91) |
| 15 | min | 7.40 (1.02) | 6.77 | (1.55) | 13.86 | (2.59) |
| 30 | min | 6.12 (1.72) | 13.03 | (3.03) | 15.35 | (2.94) |
| 45 | min | 5.07 (1.14) | 11.12 | (2.12) | 13.34 | (1.78) |
| 60 | min | 2.59 (0.47) | 11.69 | (2.79) | 10.38 | (1.48) |
| 90 | min | 3.66 (0.47) | 10.26 | (2.76) | 8.59 | (1.53) |
| 120 | min | 3.16 (0.26) | 10.30 | (3.26) | 8.87 | (1.33) |

In interpreting the data in Table 1, one sees that ganaxolone increases the average time to withdrawal for the animals tested. An increased time to withdrawal indicates that the ganaxolone is acting to reduce the animal's sensitivity to pain and thus is useful for treating neuropathic pain.

### Example 3

### (A) Tactile and (B) Thermal Allodynia in Spinal Nerve Ligation Model

(A) L5/L6 spinal nerve ligation (SNL) was performed as described by Kim and Chung (Pain, 50,355-363(1992)). Animals (mice or rats) were anesthetized with halothane. An incision was made lateral to the lumbar spine. The right L5 and L6 spinal nerves were isolated and tightly ligated distal to the dorsal root ganglion. The incision was closed, and animals were allowed to recover for 10 days. Sham-operated animals were prepared in an identical fashion except that the spinal nerves were not ligated.

Tactile withdrawal threshold was determined as described by Chaplan et al (J. Neurosci. Methods, 53,55-63 (1994). Animals were acclimated for 30 min in suspended cages with wire mesh bottoms. The hindpaw was probed with calibrated von Frey filaments (Stoelting) applied perpendicularly to the plantar surface. A positive response was indicated by a sharp withdrawal of the paw. The 50% paw withdrawal threshold was determined by the nonparametric method of Dixon, in which the stimulus is incrementally increased until a positive response is obtained, then decreased until a negative result is observed. The protocol was repeated until three changes in behavior were determined. The maximal cut-off size values used were 15 g for rats and 3.5 g for mice. The 50% paw withdrawal threshold was determined as 10Xf+kδ/10,000, where Xf=the value of the last von Frey filament used, k=Dixon value for the positive/negative pattern, and δ=the logarithmic difference between stimuli.

Tactile allodynia response was measured 15, 30, 45, 60, 90 and 120 minutes after i.p. administration of ganaxolone (at 1 mg/kg, 3 mg/kg, 6 mg/kg or 10 mg/kg). Table 2 provides the resulting measurement of tactile allodynia response in groups of five rats per dosage level. The 10 mg/kg dose sedated four of the five rats for about one hour.

TABLE 2

| | | % Activity ± s.e.m. | | | |
|---|---|---|---|---|---|
| | | 1 mg/kg | 3 mg/kg | 6 mg/kg | 10 mg/kg |
| 15 | min | 2.83 ± 2.83 | 9.69 ± 4.56 | 26.79 ± 9.34 | 47.91 ± 21.86 |
| 30 | min | 6.35 ± 3.38 | 13.76 ± 6.54 | 30.85 ± 6.77 | 79.27 ± 18.71 |
| 45 | min | 3.20 ± 2.21 | 14.02 ± 5.14 | 47.30 ± 14.99 | 70.60 ± 18.42 |
| 60 | min | 2.41 ± 1.53 | 11.56 ± 3.01 | 34.17 ± 15.78 | 64.61 ± 18.63 |
| 90 | min | 2.41 ± 1.53 | 9.19 ± 2.67 | 26.91 ± 15.45 | 24.81 ± 9.53 |
| 120 | min | 3.06 ± 3.06 | 9.64 ± 1.63 | 9.32 ± 2.85 | 4.38 ± 1.81 |

(B) The method of Hargreaves et al. (Pain, 32, 77-88 (1988)) was used to measure the thermal withdrawal latency in SNL rats. Animals were acclimated within Plexiglas enclosures on a clear glass plate maintained at 30° C. A radiant heat source (high-intensity projector lamp) was focused onto the plantar surface of the paw. When the paw was withdrawn, a motion detector halted the stimulus and a timer. A maximal cut-off of 40 sec for rats and 30 sec for mice was used to prevent tissue damage. Differences in responses between groups were tested by using the standard ANOVA statistical test followed by post hoc testing with Student's t test with Bonferroni's correction. Significance was defined as P<0.05.

As in Example 2, the data shows that ganaxolone increases the % Activity in the SNL animals, thus indicating that ganaxolone is acting to reduce the animal's sensitivity to pain. This reductions indicates that ganaxolone is useful for treating neuropathic pain.

Thermal withdrawal response was measured 15, 30, 45, 60, 90 and 120 minutes after i.p. administration of ganaxolone (at 1 mg/kg, 3 mg/kg, 6 mg/kg or 10 mg/kg). Table 3 provides the resulting measurement of thermal withdrawal in groups of five rats per dosage level. The 10 mg/kg dose sedated four of the five rats for about one hour.

TABLE 3

| | | % Activity ± s.e.m. | | | |
|---|---|---|---|---|---|
| | | 1 mg/kg | 3 mg/kg | 6 mg/kg | 10 mg/kg |
| 15 | min | 24.59 ± 14.93 | 37.15 ± 18.52 | 79.03 ± 10.50 | 70.07 ± 18.30 |
| 30 | min | 27.99 ± 17.71 | 48.03 ± 22.45 | 70.78 ± 11.53 | 46.91 ± 14.25 |
| 45 | min | 3.98 ± 2.63 | 19.84 ± 12.97 | 43.72 ± 10.03 | 47.36 ± 12.97 |
| 60 | min | 12.62 ± 8.69 | 14.25 ± 10.87 | 60.94 ± 10.27 | 41.45 ± 10.90 |
| 90 | min | 17.40 ± 8.65 | 4.18 ± 4.18 | 42.83 ± 10.41 | 33.58 ± 7.53 |
| 120 | min | 12.13 ± 9.25 | 16.57 ± 11.76 | 10.88 ± 6.34 | 23.87 ± 4.35 |

The thermal withdrawal response was measured using the same protocol in rats receiving sham surgery. Table 4 provides the resulting measurement of thermal withdrawal in groups of five rats per dose level.

TABLE 4

| | | % Activity | | |
|---|---|---|---|---|
| | | 3 mg/kg | 6 mg/kg | 10 mg/kg |
| 15 | min | 11.87 ± 7.34 | 0.00 ± 0.00 | 24.54 ± 19.33 |
| 30 | min | 3.25 ± 2.71 | 0.00 ± 0.00 | 22.60 ± 11.64 |
| 45 | min | 3.62 ± 3.62 | 3.98 ± 3.98 | 8.03 ± 4.26 |
| 60 | min | 1.98 ± 1.98 | 2.95 ± 2.95 | 0.65 ± 0.65 |
| 90 | min | 2.22 ± 1.61 | 1.73 ± 1.73 | 0.28 ± 0.28 |
| 120 | min | 1.39 ± 1.39 | 0.41 ± 0.41 | 0.02 ± 0.02 |

The subject matter claimed is:

1. A method of treating neuropathic pain in a subject experiencing such pain by administering a therapeutically effective amount of a compound for a time sufficient to reduce the intensity of such pain in the subject, wherein the neurosteroid has the formula: 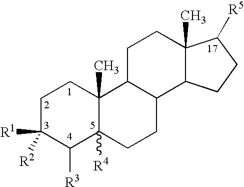
wherein
R¹ is methyl, R² is hydroxyl or a physiologically cleavable ester thereof, R³ is hydrogen, R⁴ is a hydrogen, and R⁵ is acetyl (CH₃C(O)—), and the pharmaceutically acceptable esters and salts of the hydroxyl group.

2. The method of claim 1, wherein R² is hydroxyl and R⁴ is in the alpha configuration.

3. The method of claim 1, wherein R² is hydroxyl and R⁴ is in the beta configuration.

4. The method of claim 1, wherein the compound is delivered to the subject at a rate of 0.1 mg/kg to 10 mg/kg body weight of the subject.

5. The method of claim 1 wherein the compound is ganaxolone.

6. The method of claim 1 wherein the compound is a physiologically cleavable ester of ganaxolone.

7. A composition for treating neuropathic pain in a subject experiencing such pain which comprises a pharmaceutically acceptable excipient and a therapeutically effective amount of a compound of the formula: 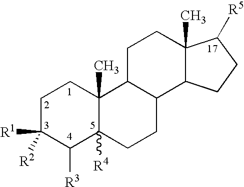
wherein
R¹ is methyl, R² is hydroxyl or a physiologically cleavable ester thereof, R³ is hydrogen, R⁴ is a hydrogen, and R⁵ is acetyl (CH₃C(O)—)

8. The composition of claim 7, wherein R² is hydroxyl and R⁴ is in the alpha configuration, i.e., ganaxolone.

9. The composition of claim 7, wherein R² is hydroxyl and R⁴ is in the beta configuration.

10. The composition of claim 7, wherein the compound is about 1% to about 90% by weight of the composition.

11. The composition of claim 7 wherein the compound is a physiologically cleavable ester of ganaxolone.

12. An article of manufacture comprising:
(a) a composition comprising a pharmaceutically acceptable excipient and compound of the formula: 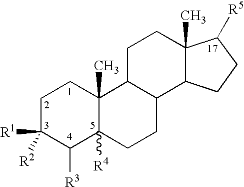
wherein
R¹ is methyl, R² is hydroxyl or a physiologically cleavable ester thereof, R³ is hydrogen, R⁴ is a hydrogen, and R⁵ is acetyl (CH₃C(O)—), and
(b) a label with instructions for using the composition to treat neuropathic pain in a patient.

13. The article of claim 12, wherein R² is hydroxyl and R⁴ is in the alpha configuration, i.e., ganaxolone.

14. The article of claim 12, wherein R² is hydroxyl and R⁴ is in the beta configuration.

15. The article of claim 12, wherein the label provides instructions to deliver the compound to the subject at a rate of 0.1 mg/kg to 10 mg/kg body weight of the subject.

16. The method of claim 12 wherein the compound is a physiologically cleavable ester of ganaxolone.

17. A method for preparing a pharmaceutical composition useful for treating neuropathic pain, which method comprises:
(a) combining a pharmaceutically acceptable excipient with a compound of the formula: 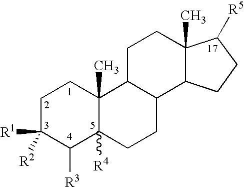
wherein
R¹ is methyl, R² is hydroxyl or a physiologically cleavable ester thereof, R³ is hydrogen, R⁴ is a hydrogen, and R⁵ is acetyl (CH₃C(O)—).
to form a formulation acceptable for administration to a human; and
(b) packaging the formulation with written instructions for the treatment of neuropathic pain by administering the formulation to a patient in need of therapy.

18. The method of claim 17, wherein R² is hydroxyl and R⁴ is in the alpha configuration, i.e., ganaxolone.

19. The method of claim 17, wherein R² is hydroxyl and R⁴ is in the beta configuration.

20. The method of claim 17, wherein the instructions indicate the compound is delivered to the subject at a rate of 0.1 mg/kg to 10 mg/kg body weight of the subject.

21. The method of claim 17 wherein the compound is a physiologically cleavable ester of ganaxolone.
